(19) United States
(12) Patent Application Publication
(10) Pub. No.: US 2026/0240571 A1
(43) Pub. Date: Aug. 20, 2026

(54) PREOPERATIVE METHOD FOR MANUFACTURING AN ARTHRODESIS DEVICE

(71) Applicant: S.M.A.I.O., SAINT PRIEST (FR)

(72) Inventor: Jean-Charles ROUSSOULY, POLEYMIEUX AU MONT D'OR (FR)

(21) Appl. No.: 19/541,814

(22) Filed: Feb. 17, 2026

(30) Foreign Application Priority Data
Feb. 17, 2025 (FR) 2501639

(51) Int. Cl. A61B 17/70; A61B 34/10

(52) U.S. Cl. CPC A61B 17/7032; A61B 17/7008; A61B 17/7037; A61B 17/7041; A61B 17/7052; A61B 2034/105

(57) ABSTRACT

The present invention relates to a preoperative method for manufacturing an arthrodesis device (10), specific to a patient, the method comprising:
a step of constructing a digital image of at least a portion of a spinal column;
a step of correcting the digital image, to form a corrected digital image;
a design step, based on the corrected digital image, of a virtual arthrodesis device;
a step of providing a plurality of real rods (12, 14) and at least one real sleeve (16);
   a step of manufacturing the arthrodesis device, wherein the real rods (12, 14) are assembled to each other via the at least one real sleeve (16) by being inserted into the at least one real sleeve (16), and the real rods (12, 14) and the at least one real sleeve (16) are fixed to each other in an irreversible manner.

### BACKGROUND

In order to treat certain spinal pathologies such as deformities, arthrodesis is often used, in a well-known manner. Arthrodesis involves implanting an arthrodesis device on the spinal column of a patient, more specifically by attaching the device to bone anchors fixed in the vertebrae of the patient, in order to partially or entirely reduce the spinal deformity. The arthrodesis device for this purpose comprises metal rods. However, it is a practitioner, for example a surgeon, who, at the time of the surgical operation of the patient, assembles the rods together, and possibly modifies their geometry, for example by bending them, so that they match the anatomy and the pathology of the patient that is to be corrected. Such a surgical operation is therefore long and complex, and requires experience on the part of the practitioner, especially since the assembly of the device is carried out during the surgical operation, which means it must be performed within limited time.

Moreover, the rods are assembled to each other via screwed connectors, which requires a large number of parts, as well as complex actions for the practitioner, who must insert the rods, possibly after having bent them, into the sleeve, screw the sleeve, then implant the arthrodesis device on the spinal column of the patient. This also implies a significant bulk of the device, caused by the screws, and a risk of loosening once the device is implanted, which reduces the reliability of the device. Finally, positioning these connectors is not always easy or even possible due to the space often being limited between the bone anchors to which the rods are attached.

It is known to use medical imaging and computer-aided design techniques in order to anticipate the length of the rods, any possible bending to be carried out on the rods in order to adapt to the patient and thus save time during the surgical operation. However, it remains necessary to limit the time spent by the practitioner assembling the arthrodesis device, and to solve the problems of bulk and potential loosening generated by the use of screws.

### SUMMARY

The aim of the invention is therefore to propose a method for manufacturing an arthrodesis device in a simple manner, which is compact and more reliable, while being specific to the patient in the body of whom it is intended to be implanted.

To this end, the invention has as its object, a preoperative method for manufacturing an arthrodesis device specific to the patient, the device comprising a plurality of real rods and at least one real sleeve, the method comprising:
a step of constructing a digital image of at least a portion of the spinal column of the patient, by means of a medical imaging apparatus;
a step of correcting the digital image, to form a corrected digital image;
a design step, based on the corrected digital image, of a virtual arthrodesis device comprising a plurality of virtual rods and at least one virtual sleeve, the virtual rods being assembled via the at least one virtual sleeve, the design step being computer-aided;
a step of providing the plurality of real rods and the at least one real sleeve;
a step of manufacturing the arthrodesis device, each real rod being associated with one of the virtual rods, so that each real rod corresponds to the virtual rod with which it is associated, in which the real rods are assembled to each other via the at least one real sleeve by being inserted into the at least one real sleeve, and the real rods and the at least one real sleeve are fixed to each other in an irreversible manner.

By means of the invention, the arthrodesis device is adapted to the patient, notably because the use of the digital image of at least a portion of the spinal column of the patient allows to design the virtual arthrodesis device by taking into account the specificities of the patient, for example anatomical specificities, and the pathology to be corrected. In addition, the preoperative manufacturing method described allows to combine real rods of different diameters easily, and thus to better adapt to the specificities of each patient.

By means of the invention, the arthrodesis device is entirely manufactured preoperatively. This allows to save time for the practitioner during the surgical operation, since they only have to implant the device, and avoids errors at the time of assembly, since they no longer need to assemble it themselves, nor possibly bend the real rods. Finally, preoperative assembly allows to achieve an irreversible fixation between the real rods and the real sleeve, which could not be performed in the operating room. This thus allows to dispense with screws, reducing bulk and improving the reliability of the device, since there is no longer any risk of the device loosening once it is implanted. This also simplifies the operation for the surgeon, who no longer has to screw the sleeves and the real rods together.

According to other advantageous aspects of the invention, the method comprises one or more of the following features, taken alone or according to any technically possible combination:
during the design step, at least one of the virtual rods is bent;
the plurality of real rods provided during the providing step is a plurality of raw real rods; and
the manufacturing step comprises a sub-step of bending at least one raw real rod, to form at least one bent real rod, so that the or each bent real rod(s) correspond(s) to the or one of the bent virtual rod(s) with which it is associated, and the or each bent real rod(s) and the raw real rods are assembled via the at least one real sleeve during an assembly sub-step;
at least one real sleeve comprises a blind hole that comprises a bottom, and during the manufacturing step, one of the real rods of the plurality of real rods is inserted into the blind hole of the at least one real sleeve so as to be in abutment against the bottom;
the blind hole is a first blind hole and the at least one real sleeve comprises a second blind hole that comprises a bottom, and during the manufacturing step, one of the real rods of the plurality of real rods is inserted into each blind hole so as to be in abutment against their respective bottoms;
the blind holes extend parallel to one another, the two real rods of the plurality of real rods respectively inserted into the blind holes have identical geometry, and during the manufacturing step, each of the two real rods is inserted into one of the blind holes so that the two real rods are parallel to each other;
at least one real sleeve comprises a bore and during the manufacturing step, one of the real rods of the plurality of real rods is inserted into the bore so as to pass through the at least one real sleeve from end to end via the bore;
at least one real sleeve comprises a bore, and during the manufacturing step, two real rods of the plurality of real rods are inserted into the bore and are in abutment against each other;
the two real rods have distinct diameters, and the bore comprises a shoulder, and during the manufacturing step, the real rod of larger diameter, called the first rod, is inserted into the bore to be in abutment against the shoulder of the bore, and the real rod of smaller diameter, called the second rod, is inserted into the bore to be in abutment against the first rod;
the two real rods have distinct diameters, the real rod of larger diameter is called the first rod, and the real rod of smaller diameter is called the second rod, and in which:
the first rod comprises a main part and an insertion end, the diameter of the first rod being the diameter of the main part, a diameter of the insertion end being less than the diameter of the first rod, the first rod comprising a shoulder at a junction between the main part and the insertion end;
the at least one real sleeve comprises a main diameter, equal to the diameter of the first rod; and
   during the manufacturing step, the insertion end is inserted into the at least one real sleeve so that the shoulder of the first rod is put into abutment against the at least one real sleeve, so that the main part and the at least one sleeve are flush relative to each other;
the bore is a first bore and the at least one sleeve comprises a second bore, the bores extending parallel to one another, and during the manufacturing step, at least one real rod is inserted into each bore;
the arthrodesis device comprises the plurality of real rods and a plurality of real sleeves, and in which during the manufacturing step, the real rods are assembled via the plurality of real sleeves in such a way that at least two real rods of the plurality of real rods are inserted into each real sleeve of the plurality of real sleeves in order to form the arthrodesis device; and
the real rods and the at least one real sleeve are fixed to each other in an irreversible manner according to a method from among the following methods: welding, brazing, bonding, press-fit assembly, and crimping.

A method for correcting a pathology of a spinal column of the patient is also described, comprising the preoperative manufacturing method described above and a step of surgical operation on the patient, in which the arthrodesis device manufactured during the preoperative method for manufacturing is implanted in the body of the patient by a practitioner.

### BRIEF DESCRIPTION OF THE DRAWINGS

The invention will appear more clearly upon reading the description that follows, given solely by way of non-limiting example, and made with reference to the drawings in which:

FIG. 1 is a perspective view of an arthrodesis device according to a first embodiment;

FIG. 2 is a cross-section of the arthrodesis device of FIG. 1;

FIG. 3 is a perspective view of an arthrodesis device according to a second embodiment;

FIG. 4 is a perspective view of an arthrodesis device according to a third embodiment;

FIG. 5 is a cross-section of an arthrodesis device according to a fourth embodiment;

FIG. 6 is a perspective view of an arthrodesis device according to a fifth embodiment, once implanted on the spinal column of a patient;

FIG. 7 is a perspective view of an arthrodesis device according to a sixth embodiment, once implanted on the spinal column of a patient;

FIG. 8 is a cross-section of an arthrodesis device according to a seventh embodiment;

FIG. 9 is a cross-section of an arthrodesis device according to an eighth embodiment;

FIG. 10 is a cross-section of an arthrodesis device according to a ninth embodiment;

FIG. 11 is a cross-section of an arthrodesis device according to a tenth embodiment; and

FIG. 12 is a flowchart of a preoperative method for manufacturing a patient-specific arthrodesis device according to the invention.

### DETAILED DESCRIPTION

Throughout the description, “equal to” is understood to mean an equality relationship, subject to manufacturing and/or assembly tolerances.

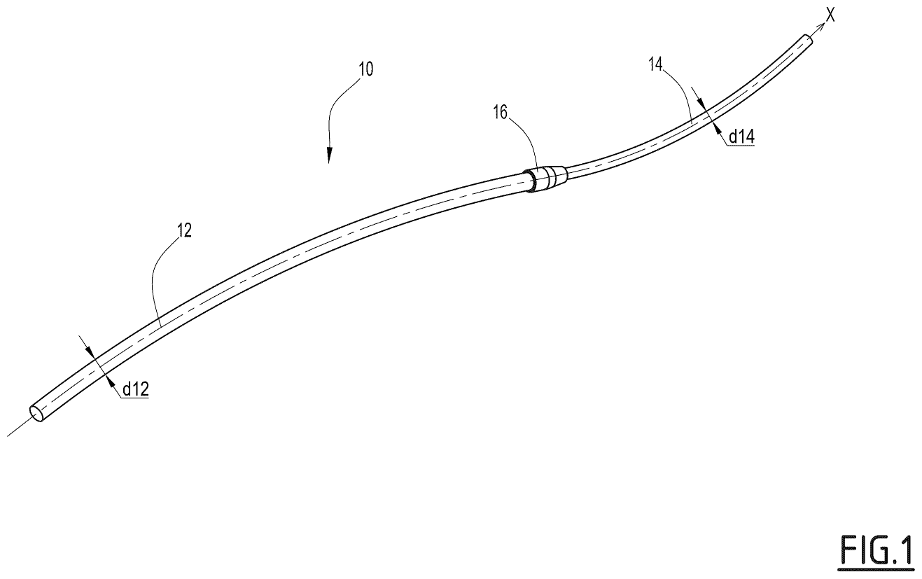

FIG. 1 represents an arthrodesis device 10, obtained according to a method for manufacturing according to the invention. The arthrodesis device 10 comprises a plurality of rods, here two rods, 12 and 14, and at least one real sleeve 16.

In the following, a rod is to be understood as a real rod, as opposed to virtual rods, which will be described below, and a sleeve is to be understood as a real sleeve.

The rods 12 and 14 are cylindrical. The rods 12 and 14, as well as the sleeve 16, are metallic, for example titanium, a chromium-cobalt alloy, or, as an alternative, a biocompatible material, for example carbon fiber, or polyetheretherketone, also called PEEK. The rod 12 is also called the first rod, and the rod 14 is also called the second rod. For example, the rods 12 and 14 and the sleeve 16 are in titanium or a cobalt-chrome alloy.

The rods 12 and 14 are, for example, in the same material, for example both in a cobalt-chrome alloy. As an alternative, one of the rods is in a cobalt-chrome alloy and the other in titanium. This allows notably to adapt the material of the rod to the stresses to which it will be subjected once implanted in the body of the patient. When the device 10 is implanted in the body of the patient, the rod 12 is for example fixed to a lumbar part of the spinal column, and the rod 14 is for example fixed to a thoracic part of the spinal column of the patient.

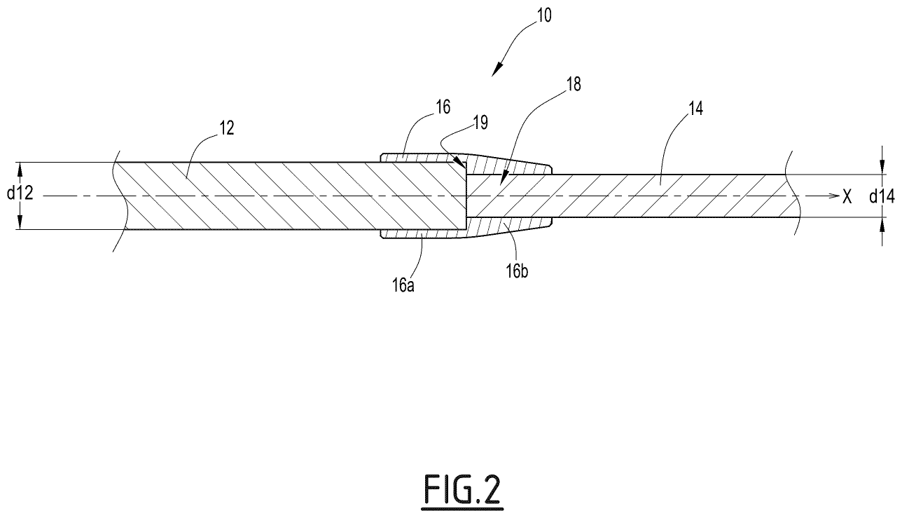

Thus, as visible in FIG. 2, the rod 12 has a diameter d12, and the rod 14 has a diameter d14, distinct from the diameter d12. Notably, the diameter d12 is greater than the diameter d14.

The rods 12 and 14 are bent, that is to say, they are curved. In the example of FIGS. 1 and 2, the rods 12 and 14 only have one curve. In an alternative, not illustrated, the rods 12 and 14 have more than one curve. The number of curves and the radius of these curvatures depend on the anatomy of the patient, as well as the pathology from which they suffer, in order to best adapt to the patient and correct their pathology. In addition, these curvatures can occur in any plane in space.

In the example of FIGS. 1 and 2, the real rods 12 and 14 are in abutment and together define a longitudinal direction X. In the example of FIGS. 1 and 2, the longitudinal direction is not rectilinear and follows the curvature of the rods 12 and 14.

The sleeve 16 comprises a tubular portion 16a and a frustoconical portion 16b. The sleeve 16 advantageously comprises a bore 18, in other words, a through-orifice. Notably the bore 18 passes through the tubular 16a and frustoconical 16b portions. The rods 12 and 14 are inserted into the sleeve 16 by being inserted into the bore 18.

In the example of FIGS. 1 and 2, the bore 18 comprises two portions with different diameters, one equal to the diameter d12, and the other equal to the d14. The bore 18 advantageously comprises a shoulder 19, advantageously at the junction between these two portions, on which the rod 12 is in abutment. The rod 14 is in abutment with the rod 12. As an alternative, the rod 14 is in contact, in other words is in abutment, against the rod 12 according to a direction opposite the longitudinal direction X.

The real rods 12 and 14 as well as the real sleeve 16 are fixed to each other, in an irreversible manner. By fixed to each other in an irreversible manner, it is understood that the real rods 12 and 14 are fixed to the real sleeve 16 by an irreversible mechanical connection, for example welding, brazing, bonding, press-fitting, or crimping. In the example of FIGS. 1 and 2, the real rods 12, 14 and the real sleeve 16 are fixed to each other via at least one laser weld.

As an alternative, not represented, an additional primary fixing is advantageously envisaged, for example by screwing the rods into the sleeve. In this case, one end of the rods 12 and 14 is threaded, and the sleeve 16 is tapped, in order to be able to screw the rods 12 and 14 inside the sleeve 16.

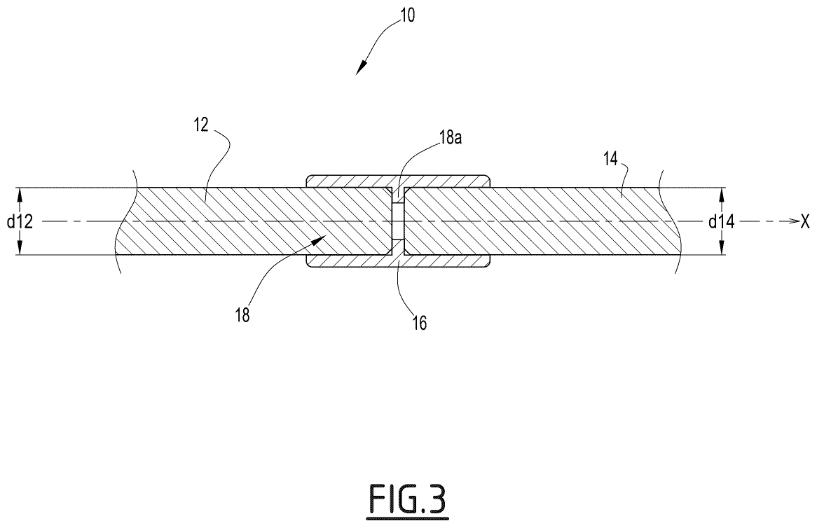

As an alternative, as well as represented in FIG. 3, the diameters d12 and d14 of the rods 12 and 14 are identical. The bore 18 comprises two portions of the same diameter, separated by a wall 18a, on which the rods 12 and 14 are in abutment.

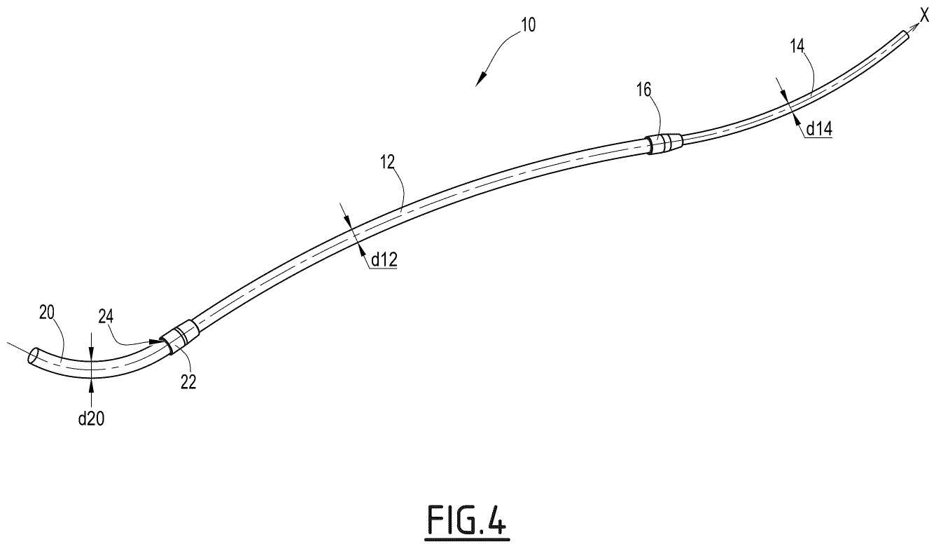

As an alternative, as well as being represented in FIG. 4, the device 10 comprises, in addition to the rods 12 and 14 and the sleeve 16, a rod 20 and a sleeve 22. The rod 20 is advantageously bent. In the example of FIG. 4, the rod 20 has a diameter d20, greater than the diameter d12.

The rod 20 is fixed to the real sleeve 22 in an irreversible manner, in a manner similar to that which has been described previously.

The sleeve 22 is for example similar to the sleeve 16 and comprises a bore 24 into which the rods 20 and 12 are inserted, and a shoulder, which is not visible in FIG. 4, against which the rod 20 is in abutment. The rod 12 is in abutment against the rod 20.

As an alternative, not represented, the sleeve 22 comprises two aligned blind holes, each blind hole comprising a bottom. One of the blind holes extends according to the longitudinal direction X, and the rod 12 is inserted into the hole and is in abutment against the bottom of the hole. The other blind hole extends according to the direction opposite the longitudinal direction X, and the rod 14 is inserted therein and is in abutment against the bottom of the hole.

As a further alternative, the device 10 comprises more than three rods and more than two sleeves.

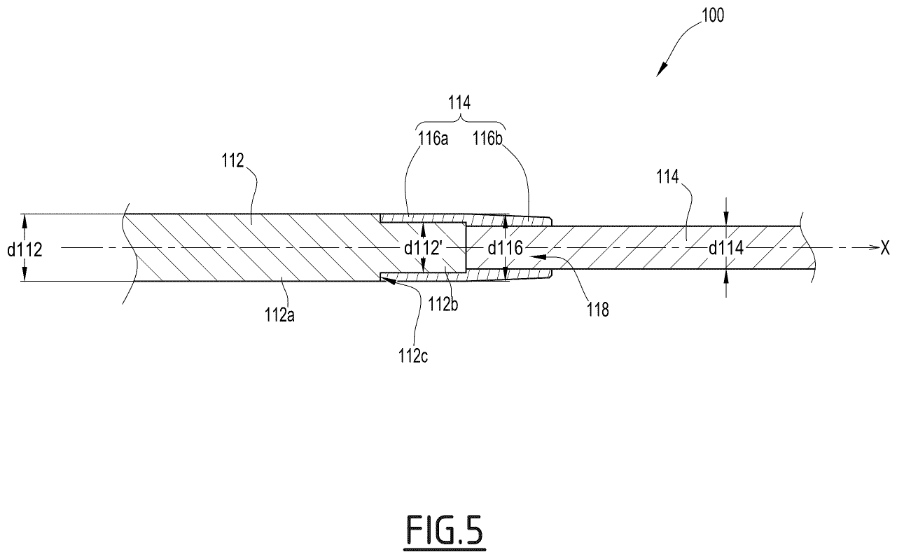

FIGS. 5 to 11 represent embodiments, alternative to the device 10. If an element is cited in the description without being shown in FIGS. 5 to 11 or if a reference is shown in a Figure without being mentioned in the description, it designates the same element as the one bearing the same reference in the first embodiment. In what follows, what mainly distinguishes these embodiments from the first is described. The number of rods described in the arthrodesis devices of FIGS. 5 to 11 is not limiting.

FIG. 5 represents a device 100 as an alternative embodiment to the device 10. The device 100 comprises a rod 112, also called the first rod, which replaces the rod 12, and a rod 114, also called the second rod, which replaces the rod 14, as well as a sleeve 116, which replaces the sleeve 116. The rods 112 and 114 extend according to the longitudinal direction X.

The rod 112 comprises a main part 112a, an insertion end 112b and a shoulder 112c at a junction between the main part 112a and the insertion end 112b. In other words, the shoulder 112c connects the main part 112a and the insertion end 112b.

The rod 112 has a diameter d112, which corresponds to the diameter of the main part 112a. The insertion end 112b has a diameter d112′, less than the diameter d112 of the main part 112a.

The rod 114 has a diameter d114, distinct from the diameter d112. In the example of FIG. 5, the diameter d114 is less than the diameter d112.

The sleeve 116 advantageously comprises a tubular portion 116a and a frustoconical portion 116b. In an alternative, not illustrated, the sleeve 116 is entirely tubular. The sleeve 116 has a main diameter d116, which corresponds to the largest diameter of the sleeve 116. In this case, this is the diameter of the tubular portion 116a.

Advantageously, and as well as being represented in FIG. 5, the main diameter d116 is equal to the diameter d112.

The sleeve 116 comprises a bore 118.

The insertion end 112b is inserted into the bore 118, so that the shoulder 112c is in abutment against the sleeve 116. Advantageously, the rod 112, more particularly the main portion 112a, and the sleeve 116, in particular the cylindrical portion 116a, are flush relative to each other. This allows, notably, to limit the bulk of the device, and to facilitate implantation of the latter in the body of the patient, because the geometry of the device 100 is as regular as possible.

The rod 114 is inserted into the bore 118 and is in abutment against the insertion end 112b. In other words, the rods 112 and 114 are in abutment

Advantageously, the bore 118 comprises a portion having a diameter equal to the diameter d112′ of the insertion end and a portion having a diameter equal to the diameter d114 of the rod 114, in order to keep the rods 112 and 114 immobile once inserted into the bore 118.

The rods 112, 114 and the sleeve 116 are fixed in an irreversible manner, for example by being welded together via one or more laser welds. Of course, as an alternative, the rods 112, 114 and the sleeve 116 are fixed together by brazing, bonding, press-fitting, or crimping.

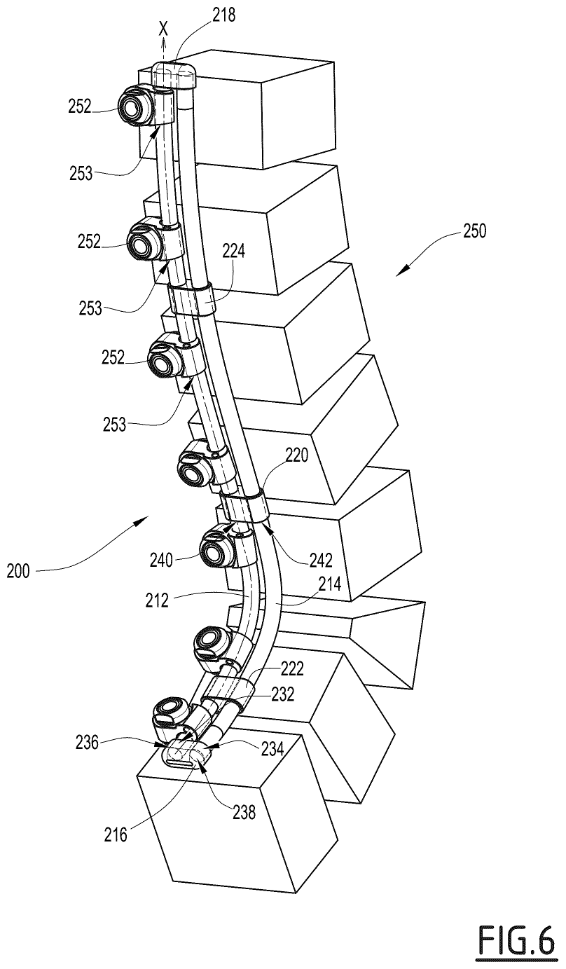

FIG. 6 is a representation of a device 200 as an alternative embodiment to the devices 10 and 100.

The device 200 comprises a rod 212 that extends according to the longitudinal direction X and a rod 214, parallel to the rod 212. The rods 212 and 214 have the same geometry, in particular an identical length and the same number of curvatures, with an identical radius of curvature, positioned at the same place along the rods. In other words, the rods 212 and 214 are interchangeable from the point of view of their geometry. The rods 212 and 214 are advantageously in the same material, or as an alternative, of different materials.

In an alternative, not represented, the rods 212 and 214 could present different diameters.

The device 200 further comprises five sleeves, 216, 218, 220, 222 and 224. The sleeves 216 and 218 have the same geometry and the sleeves 220, 222 and 224 also have the same geometry.

The sleeve 216 comprises two blind holes 232 and 234, which are parallel and extend according to the longitudinal direction X. The blind holes 232 and 234 each comprise a bottom 236, 238. The rods 112 and 114 are inserted respectively into the blind holes 232 and 234 and are in abutment against the bottoms 236 and 238, respectively.

The preceding description is applicable to the sleeve 218, with the difference that the blind holes of the sleeve 218 extend according to the direction opposite the longitudinal direction X.

The sleeves 216 and 218 are advantageously arranged on either side of the rods 212 and 214, for example to protect one end of the rods 212 and 214.

The sleeve 220 comprises two bores 240 and 242, parallel according to the longitudinal direction X. The rod 212 passes through the bore 240 from end to end and the rod 214 passes through the bore 242 from end to end. In other words, the sleeve 220 is slipped onto the rods 212 and 214 via its bores 240 and 242.

The description of the sleeve 220 is applicable to the sleeves 222 and 224.

In one alternative embodiment, not represented, sleeves identical to the sleeve 220 advantageously replace the sleeves 216 and 218 at the ends of the rods 212 and 214.

In FIG. 6, the device 200 is implanted on a spinal column 250 of the patient. For this purpose, bone anchors in the form of pedicle screws 252 are implanted on the vertebrae of the spinal column 250, and one of the rods, here rod 212, is inserted into a housing 253 of each pedicle screw 252, in order to fix the device 200 to the spinal column 250 of the patient. The housing 253 is advantageously provided in a connector of the pedicle screw 252.

The sleeves 216, 218, 220, 222 and 224 have reduced bulk, thanks to the use of an irreversible fixing such as one (or two) weld(s), for example a laser weld, brazing, bonding, press-fitting, or crimping. Thus, it is not necessary to use fixing members such as screws, which require a minimum volume to be used, which can make positioning the sleeves complex, in particular when the space between the pedicle screws 252 is reduced.

The use of two parallel rods 212, 214 and the sleeves 216, 218, 220, 222 and 224 strengthens the device 200, which allows it to undergo significant mechanical stresses without risk of breaking or deforming once the device 200 is implanted in the patient. This is the case for example if a part of a vertebra is removed, in the context of an osteotomy, which has the consequence of imposing small radii of curvature on the rods, for example less than 30 mm. This implies significant mechanical stresses in a reduced space. The reduced bulk of the device 200 and in particular of the sleeves 220, 222 and 224 then facilitates implantation of the device 200 on the spinal column 250 of the patient. Furthermore, the spacing between the two rods 212 and 214, limited to the thickness of a hook of the connector in which the housing 253 is provided, contributes to this compactness.

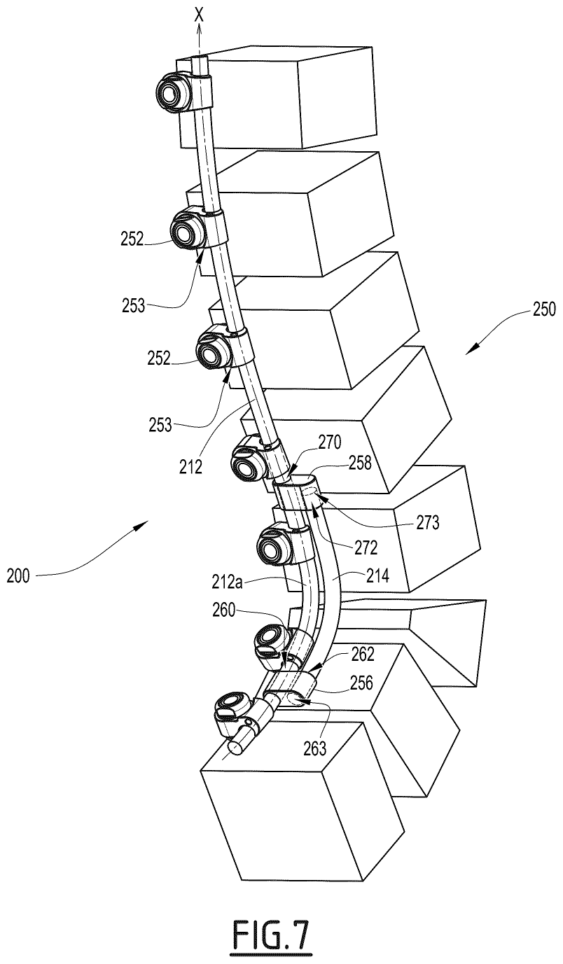

FIG. 7 represents an alternative of the device 200, in which the rods 212 and 214 have different lengths. The rod 214 is parallel to a portion 212a of the rod 212 and has the same curvature as this portion. The sleeves 216, 218, 220, 222 and 224 are replaced by two sleeves 256 and 258. The sleeve 256 comprises a bore 260 and a blind hole 262, comprising a bottom 263. The bore 260 and the blind hole 262 extend according to the longitudinal direction X and are parallel to each other. The rod 212 passes through the bore 260 from end to end and the rod 214 is inserted into the blind hole 262 so as to be in abutment against the bottom 263.

Similarly, the sleeve 258 comprises a bore 270 and a blind hole 272 comprising a bottom 273, the bore 270 and the blind hole 272 extending according to the direction opposite the longitudinal direction. The rod 212 passes through the bore 270 from end to end and the rod 214 is inserted into the blind hole 272 so as to be in abutment against the bottom 273.

In one alternative embodiment, not represented, sleeves identical to the sleeve 220 of FIG. 6 advantageously replace the sleeves 256 and 258 at the ends of the rod 214.

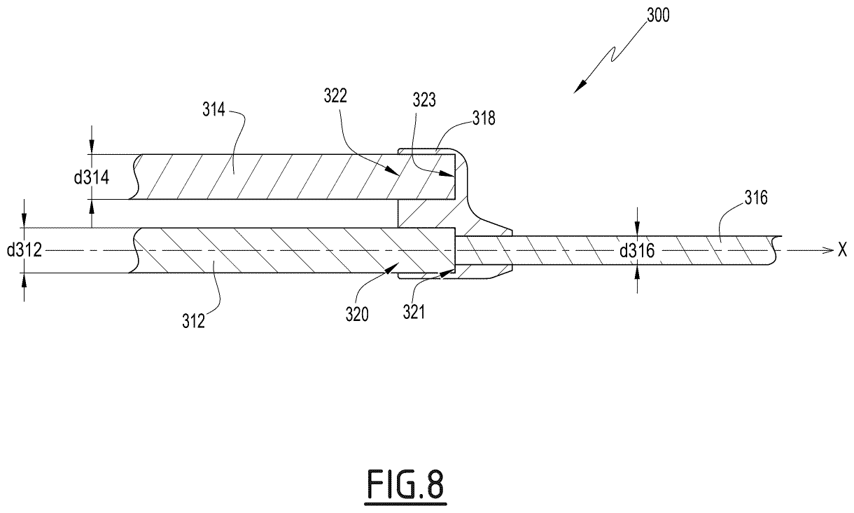

FIG. 8 represents a device 300 according to an alternative embodiment to the devices 10, 100 and 200. The device 300 comprises three rods 312, 314 and 316, and a sleeve 318. The rods 312 and 316 extend according to the longitudinal direction X, and the rods 312 and 314 are parallel to each other. The rods 312, 314, 316 are partially represented here, and are represented straight. They can of course be bent, therefore curved, in a similar manner to what has been described for the rods 12 and 14.

In the example of FIG. 8, the rods 312, 314 and 316 each have a respective diameter d312, d314 and d316. The diameters d312 and d314 are advantageously identical, and the diameter d316 is less than the diameters d312 and d314. The sleeve 318 comprises a bore 320, comprising a shoulder 321, and a blind hole 322, comprising a bottom 323. The bore 320 and the blind hole 322 are parallel to each other, and the blind hole 322 extends according to the direction opposite the longitudinal direction X.

The rod 312 is inserted into the bore 320 and is in abutment against the shoulder 321. The rod 316 is also inserted into the bore 320 and is in abutment with the rod 312 according to the direction opposite the longitudinal direction X.

The rod 314 is inserted into the blind hole 322 so as to be in abutment against the bottom 323, according to the longitudinal direction X.

As described for the previous embodiments, the rods 312, 314, 316 and the sleeve 318 are fixed to each other, for example, via an irreversible mechanical connection, for example at least one laser weld.

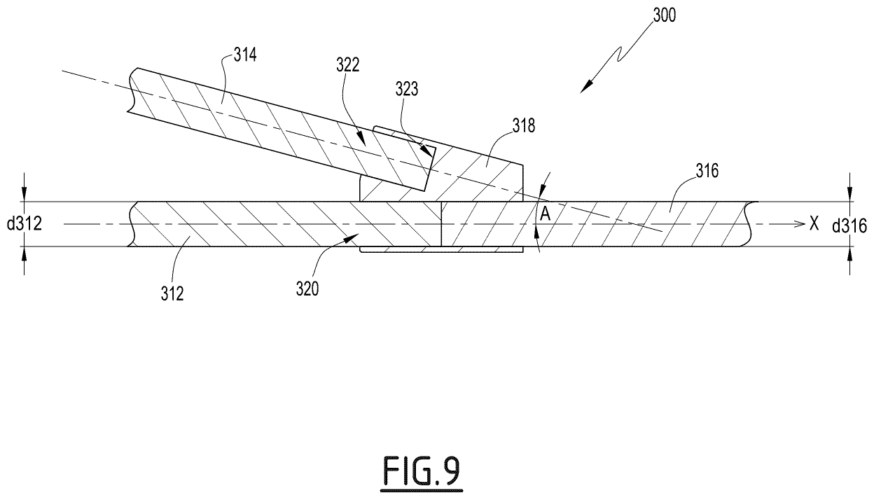

An alternative of the device 300 is represented in FIG. 9. In the example of FIG. 9, the rods 312, 314 and 316 have an identical diameter d312, d314 and d316. The blind hole 322 into which the rod 314 is inserted forms an angle A with the bore 320. The rod 314 is thus not parallel to the rod 312, and forms for example the angle A with the rod 312. The bore 320 of the sleeve 318 does not comprise a shoulder. The rods 312 and 316 are thus in abutment to each other according to the longitudinal direction X, for example by being in contact against each other according to the longitudinal direction X.

The fact that the rod 314 forms an angle A with the rod 312 facilitates, for example, the fixing of the device 300 on an iliac wing of the patient.

In FIG. 9 the three rods are in the same plane. In one alternative version, not represented, the angle A occurs in any plane in space.

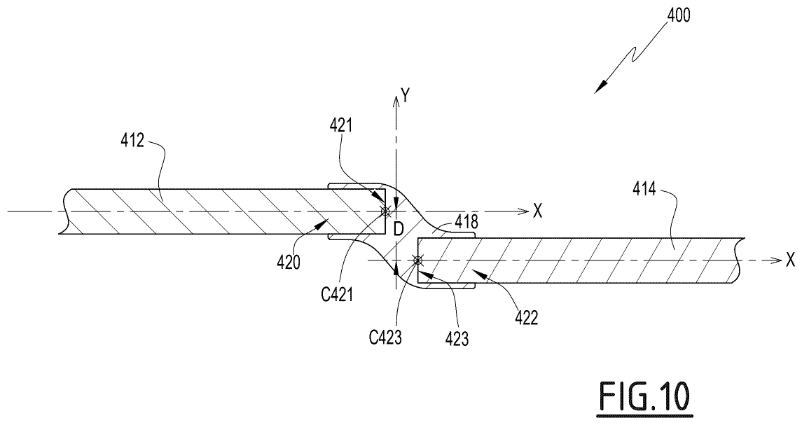

FIG. 10 represents a device 400 according to one alternative embodiment to the devices 10, 100, 200 and 300. The device 400 comprises two rods 412 and 414. The rod 412 extends according to the longitudinal direction X. The rod 414 extends according to a longitudinal direction X', which is not coincident with the longitudinal direction X. For example, the directions X and X′ are parallel, as well as represented in FIG. 10, or as an alternative, are not parallel, or even, as an alternative or in addition, are not coplanar.

The device 400 comprises a sleeve 418, comprising two blind holes 420 and 422. Each blind hole 420, 422 comprises a bottom, respectively 421 and 423.

In the example of FIG. 10, the blind holes 420 and 422 extend respectively according to the direction opposite the longitudinal direction X and according to the longitudinal direction X′. In particular, the blind holes 420 and 422 are offset relative to each other by a distance D, measured between a center C421 and C423 of each bottom 421, 423 according to a transverse direction Y, perpendicular to the directions X and X′.

The rods 412 and 414 are respectively inserted into the blind holes 420, 422 and are in abutment respectively against the bottom 421 and the bottom 423.

A sleeve 418 presenting such a shape advantageously allows to offset the rods 412, 414 according to the direction Y. Indeed, using a single rod would imply significant curvature to achieve the offset according to the transverse axis Y, and risks causing weakening of the rod. The sleeve 418, and in particular the fact that it does not require an additional fastening device, reduces the bulk needed to fix the rods 412 and 414 to each other, and allows to achieve large offsets in reduced spaces, for example when using the device 400 at the transition from the thoracic vertebrae toward the cervical vertebrae of the patient. The sleeve 418 is advantageously machined from solid.

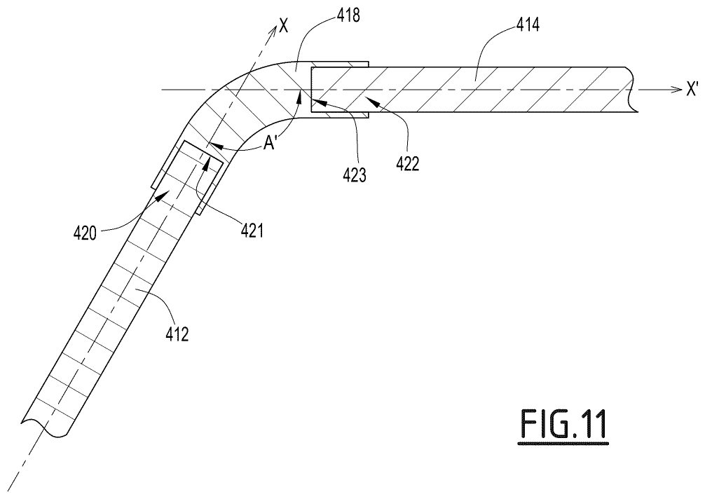

FIG. 11 is an alternative of the device 400, in which the longitudinal directions X and X′ form an angle A′ relative to each other. In this example, the sleeve 418 is curved. This allows notably to achieve a small radius of curvature of the device 400, in particular, while optimizing the mechanical strength of the latter. It is therefore possible to produce curvatures on the device 400 that would not be achievable by bending the rods themselves, without risking adversely affecting their mechanical properties.

More generally, the sleeves may comprise other combinations of bores and blind holes than those described, depending on the mechanical stresses to be supported, the specific anatomy of the patient, and the position of the device on the spinal column of the patient.

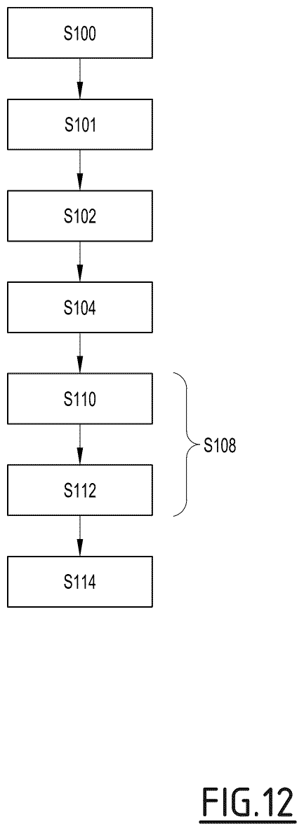

A preoperative method for manufacturing the patient-specific arthrodesis device 10 is described below, with reference to FIG. 12.

The method comprises a step S100 of constructing a digital image of at least a portion of the spinal column 250 of the patient, by means of a medical imaging apparatus. The medical imaging apparatus is for example an X-ray device or a tomography device, also called a “computer tomography scanner” in English.

The digital image thus represents the portion of the spinal column 250 of the patient.

A correction step S101 is then performed based on the digital image. The digital image is in fact corrected to form a corrected digital image. By “correction”, it is advantageously understood that a practitioner, such as a surgeon, modifies the digital image, for example by moving the vertebrae represented on the digital image. This allows to correct the portion of spinal column represented on the digital image, to reduce or eliminate excessive deformities, to correct the sagittal alignment of the spine or more generally, to limit pathological positioning of the vertebrae represented relative to each other. The corrected image thus advantageously represents a portion of the spinal column the deformation of which is limited, to approach the shape of a healthy spinal column.

Advantageously, the digital image is used to determine a positioning of the bone anchors in the vertebrae of the portion of the spinal column 250 of the patient. The practitioner chooses, for example, on which vertebrae they choose to position a bone anchor.

A design step S102 is then performed based on the corrected digital image. During the design step S102, a virtual arthrodesis device is designed in a computer-aided manner. The virtual arthrodesis device comprises a plurality of virtual rods, here two virtual rods, and at least one virtual sleeve, the virtual rods being assembled via the virtual sleeve. For example, the practitioner, based on the corrected digital image, chooses the number of rods, their length, the material, and the manner in which the virtual rods and the sleeve are assembled so as to best adapt to the anatomy of the patient and to best correct their pathology. As an alternative, the number of rods and their characteristics are determined automatically.

Advantageously, the manner in which the rods and the at least one virtual sleeve are assembled also takes into account the chosen positioning of the bone anchors, notably to avoid the available space left by the bone anchors being insufficient to accommodate a sleeve. For example, the position of the at least one virtual sleeve, and where there are several, a possible distribution of the virtual sleeves along the virtual rods, is determined, for example by the practitioner, or automatically, depending on the position of the bone anchors. The distribution is chosen for example in such a manner as to balance mechanical loads, in an arthrodesis device manufactured and implanted from the virtual device, without impinging on the bone anchors.

Advantageously, during the design step S102, at least one of the virtual rods is bent. Here, both virtual rods are bent. For example, the practitioner chooses the number of curves and their radius of curvature, so as to adapt the virtual rods to the anatomy as well as to the specific pathology of the patient.

A providing step S104 of the plurality of real rods 12 and 14, and of the real sleeve 16, is then carried out. The real rods 12, 14 are so-called raw rods, which is to say not bent, or in other words straight rods.

A manufacturing step S108 of the arthrodesis device 10 is then carried out. During the manufacturing step S108, each real rod 12, 14 is associated with one of the virtual rods, so that each real rod 12, 14 corresponds to the virtual rod with which it is associated. The real rods 12, 14 are assembled to each other via the real sleeve 16 by being inserted into the real sleeve 16, and the real rods 12, 14 and the real sleeve 16 are fixed to each other in an irreversible manner.

More precisely, the manufacturing step S108 advantageously comprises a bending sub-step S110 of the raw real rods 12, 14, to form at least, here two, bent real rods 12, 14, so that each bent real rod 12, 14 corresponds to the or one of the bent virtual rods with which it is associated.

The two real rods 12, 14, now bent, are then assembled via the real sleeve 16, advantageously during an assembly sub-step S112.

As an alternative, not represented, only one of the virtual rods is bent, and during the bending sub-step S110, only one of the raw real rods is bent, the bent real rod corresponding to the bent virtual rod with which it is associated.

The irreversible fixing is advantageously carried out according to a method from among the following methods: welding, brazing, bonding, press-fit assembly, and crimping. Preferably, the method used is laser welding.

During the assembly sub-step S112, the rod 12 is inserted into the bore 18 to be brought into abutment against the shoulder 19, and the rod 14 is then inserted into the bore 18 to be in abutment against the rod 12. The rods 12, 14 and the sleeve 16 are then fixed together in an irreversible manner.

As an alternative, the virtual arthrodesis device comprises a plurality of virtual rods, for example three, and a plurality of virtual sleeves, for example two.

The providing step S104 then comprises the provision of two real sleeves 16 and 22 and the rods 12, 14 and 20. The rods 12, 14 and 20 are bent during the bending sub-step S110. During the assembly sub-step, the rod 20 is advantageously inserted into the bore 24, then the rod 12 is inserted into the bore 24 to be in abutment against the rod 20. The rod 12 is inserted into the bore 18 to be brought into abutment against the shoulder 19, and the rod 14 is then inserted into the bore 18 to be in abutment against the rod 12.

The rods 12, 14, 20 and the sleeves 16 and 22 are then fixed to each other in an irreversible manner, to form the arthrodesis device 10 according to FIG. 4.

As an alternative, the order of assembly of the rods 12, 14, 20 and the sleeves 18 and 22 is different from that described previously, and fixing advantageously takes place progressively during assembly. For example, the rod 20 is inserted into the bore 24 and the rod 20 and the sleeve 22 are welded together before proceeding with the rest of the assembly.

In an advantageous manner, the manufacturing step S108 is carried out entirely in a factory, or in a workshop. Preferably, the manufacturing step S108 is carried out at least one day before the start of a surgical operation to implant the device in the patient. Preferably, the manufacturing step S108 is carried out by one or more people who do not participate in the operation of the patient, and the manufacturing step S108 aims to provide an arthrodesis device ready to be implanted in the body of the patient by the practitioner, or by the medical staff participating in the surgical operation. In particular, in an advantageous manner, the bending sub-step S110 is carried out by automated industrial means, as opposed to manual bending carried out by an operator or by the practitioner.

Advantageously, the preoperative method for manufacturing is part of a method for correcting a pathology of a spinal column of the patient. The correction method comprises the preoperative method for manufacturing described above, followed by a patient surgical operation step S114. During the operation step S114, the arthrodesis device 10 manufactured during the preoperative manufacturing method is implanted in the body of the patient by a practitioner, for example a surgeon, in order to correct the pathology from which the patient suffers. For example, during this surgical operation step S114, pedicle screws 252 are implanted on at least part of the vertebrae of the spinal column 250 of the patient, and the device 10 is fixed to these pedicle screws 252.

Generally, two devices are implanted in the body of the patient, one on each side of the spinal column 250. However, as an alternative, only one device or more than two devices are implanted in the body of the patient.

The method for manufacturing the devices 100, 200, 300 and 400 is identical to that described above, except for the differences described below.

In the preoperative method for manufacturing an arthrodesis device 100, the rods 112, 114 and the sleeve 116 are provided during the providing step S104.

The rods 112 and 114 are advantageously bent during the bending sub-step S110.

During the manufacturing step S108, in particular during the assembly sub-step S112, the insertion end 112b is inserted into the sleeve 116 so that the shoulder 112c is brought into abutment against the sleeve 116, so that the main part 112 and the sleeve 116 are flush relative to each other.

In the preoperative method for manufacturing an arthrodesis device 200, the rods 212 and 214 as well as the sleeves 216, 218, 220, 222 and 224 are provided during the providing step S104.

Advantageously, the rods 212 and 214 are bent during the bending sub-step S110.

During the assembly sub-step S112, the rods 212 and 214 are inserted respectively into the blind holes 232 and 234 of the sleeve 216, so as to be in abutment respectively against the bottoms 236 and 238 respectively, and so that the rods 212 and 214 are parallel to each other. The same applies to the sleeve 218.

The rods 212 and 214 are respectively inserted into the bores 240 and 242 of the sleeve 220, so as to pass through the sleeve 220 from end to end via the bores 240 and 242. The same applies to sleeves 222 and 224. Thus, a real rod 212, 214 is inserted into each bore 240, 242.

As an alternative, assembly of the rods 212, 214 and the sleeves 216, 218, 220, 222 and 224 takes place in a different order, notably the rods 212 and 214 are inserted into the sleeve 218 after the sleeves 220, 222 and 224.

The irreversible fixing of the rods 212, 214 and sleeves 216, 218, 220, 222 and 224 takes place at the same time as assembly, or once the rods 212, 214 are inserted into all of the sleeves 216, 218, 220, 222 and 224. The device 200 according to FIG. 5 is then manufactured and is ready to be implanted in the body of the patient.

As an alternative, during the providing step, the rods 212 and 214 of different sizes and the sleeves 256 and 258 are provided during the providing step S104.

The rod 212 is inserted into the bore 260 of the sleeve 262, so as to pass through the sleeve 256 from end to end via the bore 260. The rod 212 is inserted into the blind hole 262 so as to be brought into abutment against the bottom 263, and to be parallel to the portion 212a. The same applies to the sleeve 258. The rods 212, 214 and the sleeves 256, 258 are fixed to each other in an irreversible manner, simultaneously with assembly, or once the rods 212, 214 are inserted into both the sleeves 256, 258.

In the preoperative method for manufacturing the arthrodesis device 300, the rods 312, 314, 316 and the sleeve 318 are provided during the providing step S104.

Advantageously, the rods 312, 314 and 316 are bent during the bending sub-step S110.

Advantageously, during the assembly sub-step S112, the rod 312 is inserted into the bore 320 to be brought into abutment against the shoulder 321 and the rod 316 is then inserted into the bore 320 to be in abutment against the rod 312. As an alternative, if the rods 312 and 316 have the same diameter, they are simply inserted into the bore 320 and brought into abutment to each other, for example by being brought into contact with each other.

The rod 314 is inserted into the blind hole 322 to be brought into abutment against the bottom 323. The rod 314 is thus parallel to the rod 312, or as an alternative, forms the angle A with the rod 312, depending on the orientation of the blind hole 322.

The rods 312, 314, 316 and the sleeve 319 are fixed in an irreversible manner to each other.

In the preoperative method for manufacturing the arthrodesis device 400, the rods 412, 414 and the sleeve 418 are provided during the providing step S104. The rods 412 and 414 are inserted into each of the blind holes 420, 422, so as to be in abutment against their respective bottoms 421, 423.

Advantageously, in the case where the real sleeve comprises two bores extending parallel to one another, during the manufacturing step S108, and in particular during the assembly sub-step S112, at least one real rod is inserted into each bore.

Any feature described for one embodiment or an alternative in the above can be implemented for the other embodiments and alternatives described previously, provided it is technically feasible.

1. A preoperative method for manufacturing a patient-specific arthrodesis device, the device comprising a plurality of real rods and at least one real sleeve, the method comprising:
a step of constructing a digital image of at least a portion of a spinal column of the patient, by means of a medical imaging apparatus;
a step of correcting the digital image, to form a corrected digital image;
a design step, based on the corrected digital image, of a virtual arthrodesis device comprising a plurality of virtual rods and at least one virtual sleeve, the virtual rods being assembled via the at least one virtual sleeve, the design step being computer-aided;
a providing step of the plurality of real rods and of the at least one real sleeve;
a manufacturing step of the arthrodesis device, each real rod being associated with one of the virtual rods, so that each real rod corresponds to the virtual rod with which it is associated, wherein the real rods are assembled to each other via the at least one real sleeve by being inserted into the at least one real sleeve, and the real rods and the at least one real sleeve are fixed to each other in an irreversible manner.

2. The method according to claim 1, wherein:
during the design step, at least one of the virtual rods is bent;
the plurality of real rods provided during the providing step is a plurality of raw real rods; and
the manufacturing step comprises a bending sub-step of at least one raw real rod, to form at least one bent real rod, so that the or each bent real rod(s) correspond(s) to the or each bent virtual rod(s) with which it is associated, and the or each bent real rod(s) and the raw real rods are assembled via the at least one real sleeve during an assembly sub-step.

3. The method according to claim 1, wherein the at least one real sleeve comprises a blind hole that comprises a bottom, and during the manufacturing step, one of the real rods of the plurality of real rods is inserted into the blind hole of the at least one real sleeve, so as to be in abutment against the bottom.

4. The method according to claim 3, wherein the blind hole is a first blind hole and the at least one real sleeve comprises a second blind hole that comprises a bottom, and during the manufacturing step, one of the real rods of the plurality of real rods is inserted into each blind hole so as to be in abutment against their respective bottoms.

5. The method according to claim 4, wherein the blind holes extend parallel to one another, the two real rods of the plurality of real rods respectively inserted into the blind holes have identical geometry, and during the manufacturing step, each of the two real rods is inserted into one of the blind holes so that the two real rods are parallel to each other.

6. The method according to claim 1, wherein the at least one real sleeve comprises a bore and during the manufacturing step, one of the real rods of the plurality of real rods is inserted into the bore so as to pass through the at least one real sleeve from end to end via the bore.

7. The method according to claim 1, wherein the at least one real sleeve comprises a bore, and during the manufacturing step, two real rods of the plurality of real rods are inserted into the bore and are in abutment.

8. The method according to claim 7, wherein the two real rods have distinct diameters and the bore comprises a shoulder, and during the manufacturing step, the real rod of larger diameter, called the first rod, is inserted into the bore to be brought into abutment on the shoulder of the bore, and the real rod of smaller diameter, called the second rod, is inserted into the bore to be in abutment with the first rod.

9. The method according to claim 7, wherein the two real rods have distinct diameters, the real rod of larger diameter is called the first rod, and the real rod of smaller diameter is called the second rod, and wherein:
the first rod comprises a main part and an insertion end, the diameter of the first rod being the diameter of the main part, a diameter of the insertion end being less than the diameter of the first rod, the first rod comprising a shoulder at a junction between the main part and the insertion end;
the at least one real sleeve comprises a main diameter, equal to the diameter of the first rod; and
during the manufacturing step, the insertion end is inserted into the at least one real sleeve so that the shoulder of the first rod is brought into abutment against the at least one real sleeve, so that the main part and the at least one sleeve are flush relative to each other.

10. The method according to claim 6, wherein the bore is a first bore and the at least one sleeve comprises a second bore, the bores extending parallel to one another, and during the manufacturing step, at least one real rod is inserted into each bore.

11. The method according to claim 1, wherein the arthrodesis device comprises the plurality of real rods and a plurality of real sleeves, and wherein during the manufacturing step, the real rods are assembled via the plurality of real sleeves in such a way that at least two real rods of the plurality of real rods are inserted into each real sleeve of the plurality of real sleeves in order to form the arthrodesis device.

12. The method according to claim 1, wherein the real rods and the at least one real sleeve are fixed to each other in an irreversible manner according to a method from among the following methods: welding, brazing, bonding, press-fit assembly, and crimping.
